(19) United States
(12) Patent Application Publication
(10) Pub. No.: US 2023/0003519 A1
(43) Pub. Date: Jan. 5, 2023

(54) DEVICE FOR MEASURING AMOUNT OF PROBE DISPLACEMENT USING CHANGE IN AMOUNT OF LIGHT

(71) Applicant: NEWPONG Co., Ltd, Wonju-si (KR)

(72) Inventor: Jaeyong LEE, Seoul (KR)

(21) Appl. No.: 17/831,149

(22) Filed: Jun. 2, 2022

(30) Foreign Application Priority Data
Jul. 1, 2021 (KR) 10-2021-0086438

(51) Int. Cl. G01C 3/02; G01R 13/00

(52) U.S. Cl. CPC G01C 3/02; G01R 13/00; A61B 8/4254

(57) ABSTRACT

Provided is a device for measuring the amount of probe displacement using a change in amount of light, the device including: a sensor mounting unit having a sensor and configured to adjust a position so that a probe is provided at a position corresponding to the sensor; a handpiece fixing unit configured to fix a handpiece by means of a through-hole formed at a center of the handpiece fixing unit; and an impedance matching unit configured to generate acoustic impedance to the probe.

### BACKGROUND

### Field

The present application relates to a device for measuring the amount of probe displacement using a change in amount of light.

### Description of the Related Art

An end of a probe of an ultrasonic surgical device needs to measure amplitude (displacement) that is changed by vibration.

Because an ultrasonic frequency is 30 kHz to 40 kHz and the displacement is about 20 um to 150 um, it is significantly difficult to check the amplitude (displacement) with the naked eye or through images.

A measurement method using a photo interrupt sensor is generally used to measure the amplitude (displacement). There is used a sensor with the specifications in which a reaction speed is 10 usec or less and there is a section in which a distance linearly varies by about 1000 um depending on the amount of light.

The background art of the present application is disclosed in Korean Patent Application Laid-Open No. 10-2014-0036650.

### SUMMARY

An object to be achieved by the present disclosure is to provide a device for measuring the amount of probe displacement using a change in amount of light, which is capable of measuring amplitude (displacement) of a probe that cannot be checked with the naked eye and through an image.

However, technical problems to be solved by the exemplary embodiment of the present application are not limited to the aforementioned technical problem, and other technical problems may be present.

According to an aspect of the present disclosure, there is provided a device for measuring the amount of probe displacement using a change in amount of light, the device including: a sensor mounting unit having a sensor and configured to adjust a position so that a probe is provided at a position corresponding to the sensor; a handpiece fixing unit configured to fix a handpiece by means of a through-hole formed at a center of the handpiece fixing unit; and an impedance matching unit configured to generate acoustic impedance to the probe.

According to the embodiment of the present application, the device may further include a measurement unit configured to measure a change in voltage by using an oscilloscope after converting a signal outputted from the sensor by using a removing circuit and an amplifying circuit.

According to the embodiment of the present application, the measurement unit may calculate the amount of displacement of the probe by converting the measured voltage into a distance.

According to the embodiment of the present application, the impedance matching unit may generate acoustic impedance to the probe by using a sponge absorbing moisture.

According to the embodiment of the present application, the sensor may be a photo interrupt sensor that has a section in which a preset distance linearly varies depending on the amount of light and a feature in which a reaction speed is a reference time or less.

The technical solution is just illustrative but should not be interpreted as being intended to limit the present application. In addition to the above-mentioned exemplary embodiment, additional exemplary embodiments may be present in the drawings and the detailed description of the invention.

According to the technical solutions according to the present application, it is possible to measure the amplitude (displacement) of the probe that cannot be checked with the naked eye and through images.

However, the effects, which can be obtained by the present application, are not limited to the above-mentioned effects, and other effects may be present.

### BRIEF DESCRIPTION OF THE DRAWINGS

The above and other aspects, features and other advantages of the present disclosure will be more clearly understood from the following detailed description taken in conjunction with the accompanying drawings, in which:

FIG. 1 is a schematic configuration view of a device for measuring the amount of probe displacement using a change in amount of light according to an embodiment of the present application; and

FIG. 2 is a view for explaining information on detection position characteristics, response time, and load resistance characteristics of a photo interrupt sensor according to the embodiment of the present application.

### DETAILED DESCRIPTION OF THE EMBODIMENT

Hereinafter, exemplary embodiments of the present application will be described in detail with reference to the accompanying drawings so that those with ordinary skill in the art to which the present application pertains may easily carry out the exemplary embodiments. However, the present application may be implemented in various different ways, and is not limited to the exemplary embodiments described herein. A part irrelevant to the description will be omitted in the drawings in order to clearly describe the present application, and similar constituent elements will be designated by similar reference numerals throughout the specification.

Throughout the specification of the present application, when one constituent element is referred to as being “connected to” another constituent element, one constituent element can be “directly connected to” the other constituent element, and one constituent element can also be “electrically connected to” or “indirectly connected to” the other element with other elements therebetween.

Throughout the specification, when one member is disposed “on”, “at an upper side of”, “at an upper end of”, “below”, “at a lower side of”, or “at a lower end of” another member in the present specification of the present application, this includes not only a case where one member is brought into contact with another member, but also a case where still another member is present between the two members.

Throughout the specification of the present application, unless explicitly described to the contrary, the word “comprise” or “include” and variations, such as “comprises”, “comprising”, “includes” or “including”, will be understood to imply the inclusion of stated constituent elements, not the exclusion of any other constituent elements.

An ultrasonic surgical device to be described below may be a device configured to come into contact with a part of a patient's body, acquire ultrasonic image information, and generate ultrasonic waves on the basis of a control signal. In addition, the ultrasonic surgical device (ultrasonic device) may be used for diagnostic purposes using functions such as structure imaging of organs and tissue in a human body, blood flow information and imaging, and characteristics and functional analysis of tissue by irradiating the interior of the human body with ultrasonic waves.

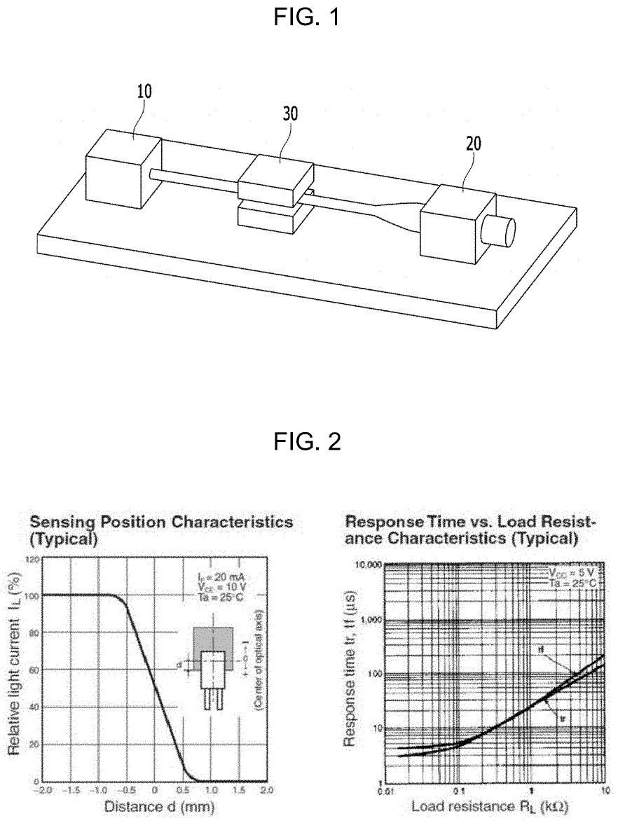

FIG. 1 is a schematic configuration view of a device for measuring the amount of probe displacement using a change in amount of light according to an embodiment of the present application.

Hereinafter, for the convenience of description, a device 1 for measuring the amount of probe displacement using a change in amount of light will be referred to as the present device 1.

The present device 1 may measure amplitude (displacement) by which an end of a probe of the ultrasonic surgical device is moved by vibration. Because it is difficult to check the amplitude (displacement) of the probe moved by the vibration with the naked eye and through images, the present device 1 may use a photo interrupt sensor to measure the amount of probe displacement.

For example, a handpiece may include an ultrasonic vibrator, a transducer, and an EEPROM. The handpiece may include the transducer configured to convert an electrical signal into mechanical vibration, and the EEPROM configured to store information such as usage time and serial numbers of the handpiece.

The handpiece may include the transducer and generate ultrasonic waves by using the ultrasonic vibrator on the basis of the control signal. The handpiece may generate a resonant frequency by receiving a signal varied within a predetermined range by supplied voltage.

The ultrasonic waves are generated by the resonant frequency from the ultrasonic vibrator. The ultrasonic vibrator may maintain an ultrasonic output by matching the varying current frequency with the resonant frequency which is a frequency at maximum voltage, thereby improving a function of an ultrasonic therapeutic device.

Referring to FIG. 1, the present device 1 may include a sensor mounting unit 10, a handpiece fixing unit 20 and an impedance matching unit 30.

According to the embodiment of the present application, the sensor mounting unit 10 may include a sensor and adjust a position of the sensor so that the probe may be provided at a position corresponding to the sensor.

For example, the sensor mounting unit 10 may determine whether the probe is provided at the position corresponding to the sensor. When the probe is not provided at the position corresponding to the sensor, the sensor mounting unit 10 may generate a control signal for controlling an operation of a motor included in the sensor mounting unit 10.

On the basis of the control signal, the motor included in the sensor mounting unit 10 may move the probe in unit of micrometer (um) along an x-axis, a y-axis, and a z-axis.

For example, the sensor may be a photo interrupt sensor. The photo interrupt sensor refers to a device capable of detecting an object, detecting a position, and counting by blocking an optical path by using a single photo coupler.

The sensor of the sensor mounting unit 10 may be a photo interrupt sensor having a section in which a preset distance (e.g., 1,000 um) linearly varies depending on the amount of light and having a reaction speed is a reference time (10 usec) or less.

In other words, as the photo interrupt sensor of the sensor mounting unit 10, there is used a sensor with the specifications in which a reaction speed is 10 usec or less and there is a section in which a distance linearly varies by about 1000 um depending on the amount of light.

According to the embodiment of the present application, the handpiece fixing unit 20 may fix the handpiece so that the handpiece is not moved. The handpiece fixing unit 20 may fix the handpiece by means of a through-hole formed in a center of the handpiece fixing unit 20.

Although not illustrated in the drawings, the handpiece fixing unit 20 may be divided into an upper end and a lower end. One end of the upper end and one end of the lower end may be coupled by means of a hinge structure. The handpiece fixing unit 20 is divided into the upper end and the lower end, and the handpiece is accommodated in the through-hole formed at the center of the handpiece fixing unit 20, such that the handpiece may be more stably fixed. However, the present application is not limited thereto.

According to the embodiment of the present application, the impedance matching unit 30 may generate acoustic impedance to the probe.

The impedance matching unit 30 generates acoustic impedance to the probe by using a sponge absorbing moisture.

The impedance matching unit 30 may be provided in the form of a sponge wet with water so as to generate proper acoustic impedance to the probe.

The ultrasonic probe includes a transducer. In this case, the transducer may include: a piezoelectric layer configured to convert an electrical signal and an acoustic signal into each other by vibrating a piezoelectric material; a matching layer configured to reduce an acoustic impedance difference between the piezoelectric layer and a test object so that ultrasonic waves generated by the piezoelectric layer are maximally transmitted to the test object; a lens layer configured to focus the ultrasonic waves, which propagate toward a front side of the piezoelectric layer, onto a particular point; and a sounding absorbing layer configured to prevent image distortion by preventing the ultrasonic waves from propagating toward a rear side of the piezoelectric layer.

As the ultrasonic transducer, a magnetostrictive ultrasound transducer using magnetostriction of a magnetic element may be used, a capacitive micromachined ultrasonic transducer configured to transmit and receive ultrasonic waves by using vibration of several hundreds or thousands of finely processes thin films may be used, or a piezoelectric ultrasonic transducer using a piezoelectric effect of a piezoelectric material may be used.

A piezoelectric effect and an inverse piezoelectric effect refer to effects in which voltage is generated by mechanical pressure applied to a predetermined material and mechanical deformation occurs because of the applied voltage. Further, the piezoelectric material refers to a material having the above-mentioned effect. That is, the piezoelectric material may be a material that converts electrical energy into mechanical vibrational energy and converts mechanical vibrational energy into electrical energy.

Meanwhile, the acoustic impedance may be one of the variables used to determine a matching condition to efficiently supply acoustic energy to a human body from the piezoelectric material.

According to the embodiment of the present application, a measurement unit (not illustrated) may use a removal circuit and an amplifying circuit and convert a signal outputted from the sensor. The measurement unit (not illustrated) may convert the signal outputted from the sensor into a measurable signal by applying the signal outputted from the sensor to a DC-off removal circuit and the amplifying circuit.

In addition, the measurement unit (not illustrated) may measure a change in voltage by applying the converted output signal to an oscilloscope.

The measurement unit (not illustrated) may calculate the amount of probe displacement by converting the measured voltage into a distance.

Meanwhile, the measured frequency may be an oscillation frequency of the ultrasonic vibrator.

It will be appreciated that the embodiments of the present application have been described above for purposes of illustration, and those skilled in the art may understand that the present application may be easily modified in other specific forms without changing the technical spirit or the essential features of the present application. Therefore, it should be understood that the above-described embodiments are illustrative in all aspects and do not limit the present disclosure. For example, each component described as a single type may be carried out in a distributed manner. Likewise, components described as a distributed type can be carried out in a combined type.

The scope of the present application is represented by the claims to be described below rather than the detailed description, and it should be interpreted that the meaning and scope of the claims and all the changes or modified forms derived from the equivalent concepts thereto fall within the scope of the present application.

What is claimed is:

1. A device for measuring the amount of probe displacement, the device comprising: a sensor mounting unit having a sensor and configured to adjust a position so that a probe is provided at a position corresponding to the sensor; a handpiece fixing unit configured to fix a handpiece by means of a through-hole formed at a center of the handpiece fixing unit; and an impedance matching unit configured to generate acoustic impedance to the probe.

2. The device of claim 1, further comprising: a measurement unit configured to measure a change in voltage by using an oscilloscope after converting a signal outputted from the sensor by using a removing circuit and an amplifying circuit.

3. The device of claim 2, wherein the measurement unit calculates the amount of displacement of the probe by converting the measured voltage into a distance.

4. The device of claim 2, wherein the impedance matching unit generates acoustic impedance to the probe by using a sponge absorbing moisture.

5. The device of claim 1, wherein the sensor is a photo interrupt sensor that has a section in which a preset distance linearly varies depending on the amount of light and a feature in which a reaction speed is a reference time or less.
